(19) United States
(12) Patent Application Publication
(10) Pub. No.: US 2024/0384270 A1
(43) Pub. Date: Nov. 21, 2024

(54) NUCLEIC ACID MOLECULE AND ITS USE

(71) Applicant: RNA MORPH LTD, Even Yehuda (IL)

(72) Inventor: Eitan LEV, Even Yehuda (IL)

(73) Assignee: RNA MORPH LTD, Even Yehuda (IL)

(21) Appl. No.: 18/576,656

(22) PCT Filed: Jul. 7, 2022
(86) PCT No.: PCT/IL2022/050735

§ 371 Date: Jan. 4, 2024

Related U.S. Application Data
(60) Provisional application No. 63/203,097, filed on Jul. 8, 2021.
(60) Provisional application No. 63/263,709, filed on Nov. 8, 2021.

(51) Int. Cl. C12N 15/113

(52) U.S. Cl. CPC C12N 15/113; C12N 2310/11; C12N 2310/315; C12N 2320/33

(57) ABSTRACT

Nucleobases-comprising complex having two nucleobase strands linked by a common backbone structure common to the two strands. The common backbone structure may be formed by coupling two backbones, one of each of the strands, by molecular (e.g., covalent) bonding or other type of bonding; or may be a single backbone structure for the two strands. The first nucleic acid strand is, typically, such that it can specifically hybridize to a nucleotide target sequence, which may be a coding DNA sequence or an mRNA sequence and is typically complementary to that sequence. The second nucleic acid strand remains exposed after hybridization and has a sequence such so as to permit interaction with certain agents in the environment.

### TECHNOLOGICAL FIELD

The present disclosure is in the field of solutions for overcoming nucleic acid mutations.

### GENERAL DESCRIPTION

The present disclosure provides novel nucleobases-comprising complex or conjugate and their use. The complex comprises two nucleobase strands linked by a common backbone structure common to the two strands. The common backbone structure may be formed by coupling two backbones, one of each of the strands, by molecular (e.g., covalent) bonding or other type of bonding: or may be a single backbone structure for the two strands.

To do that, two nucleic acid strands, not fully complementary to one another (and typically complementary but for one or a few bases), are coupled, not through their nucleobases, but rather through a backbone carrying said nucleobases, such as a sugar backbone to form the common backbone. The two backbones may be coupled to one another such that the spatial arrangement of the resulted complex allows one sequence to hybridize with another nucleic acid sequence of DNA or RNA, typically a complementary sequence thereof, while the second sequence of the complex is exposed to the cell environment, in the event that it is brought into the cell, or to another medium, where, for example, it is used in vitro, allowing it to interact with other molecules or complexes such as ribosome, spliceosome, tRNA, miRNA, etc. Thus, the complex of the present disclosure can be referred to as a back-to-back antisense oligonucleotide (bbASO; which may also be referred to herein, occasionally, as “complex”). The bbASO of this disclosure may comprise nucleotides (ribonucleotides or deoxyribonucleotides), synthetic and non-naturally occurring nucleotides, nucleic acid analogs (with a backbone-forming residue other than that found in DNA or RNA, namely other than a ribose or deoxyribose bound to a phosphate group) or any combination thereof. The non-naturally occurring nucleic acid analogs may have the effect of increasing resistance of the molecules to degradation in the extracellular and intracellular medium, while retaining the ability of one of the sequences to hybridize with another nucleic acid sequence and that of the other to interact with other molecules. Examples of nucleic acid analogs may be one of many such nucleic acid analogs that are used or proposed for use in antisense therapies. Other exemplary nucleosides may include nucleotide analogs which are building blocks of locked nucleotides (LNA or bridged nucleotides), unlocked nucleotides (UNA), s-Oligo nucleotides, peptide nucleic acid (PNA), spherical nucleic acid (SNA), trycyclo-DNA nucleotides or morpholino nucleotides.

The bbASO of this disclosure has, thus, two strands of nucleobases formed along a common backbone with the nucleobases of the two strands extending outwardly from this common backbone. The two strands are not complementary to one another. One of these strands (defined herein as “first strand”, as also noted below) has a sequence that is complementary to a certain target sequence in a DNA or RNA molecule and thus can hybridize to the target sequence. Once hybridized, the other sequence (herein the “second sequence”, as also noted below) is exposed and can have a variety of biological functions. By one embodiment, it may have a sequence that corrects a mutation in the target sequence, whereby the transcription or translation machinery, as the case may be, “see” an overall correct exposed sequence, which is a combination of the sequence of the second strand portions of the DNA or RNA, to which the first stand hybridizes, that other than the target sequence (with the mutation), e.g., portions flanking the target sequence. By another embodiment the second strand has a sequence permitting it to interact with other biological molecules, molecular complexes, cellular mechanisms, miRNA, and others. The second strand may be designed to have such a sequence to render it capable of interacting in a desired manner.

The solution provided by said one embodiment aims to (i) mask a nucleic acid mutation site (ii) and expose instead of the mutation site, a corrected, non-mutated sequence, and by that allowing normal expression of the gene associated with said mutation.

While not limited thereto, the bbASO of this disclosure may, therefore, be used for correcting genetic mutations by either complexing with the respective DNA gene sequence such that the cell's transcription mechanism would “read” a corrected nucleotide sequence and yield the transcription of a messenger RNA (mRNA) molecule with a correct, non-mutated sequence: or alternatively, complex with an mRNA molecule carrying a mutation such that the cell's transcription mechanism will “read” a corrected nucleotide sequence giving rise to the synthesis of a non-mutated protein. By some embodiments of this disclosure, one or more of the nucleic acid bases of one or both of the strands constituting the new complex of this disclosure are non-naturally occurring or nucleotide analogs, such as those exemplified above: by other embodiments one or both of the strands are constituted entirely of such bases.

The following are some terms used in the description below and their meanings within the context of this disclosure:
the terms “first nucleic acid strand” or “first strand” will be used to denote that strand with a sequence that can hybridize to the target sequence of DNA or RNA:
the terms “second nucleic acid strand” or “second strand” will be used to denote that strand that after such hybridization remains exposed to the cellular environment:
the terms “first sequence” and “second sequence” will be used to denote the sequences of the first and second strands, respectively.
the term “target sequence” will be used to denote the mutated coding DNA sequence or the mRNA sequence to which the first nucleic acid strand is intended to hybridize to; and
the term “full sequence” will be used to denote the entire DNA or RNA coding sequence that includes that target sequence:

Other terms used herein are defined and explained in the text.

The first nucleic acid strand is, typically, such that it can specifically hybridize to a nucleotide target sequence, which may be a coding DNA sequence or an mRNA sequence and is typically complementary to that sequence. The second nucleic acid strand remains exposed after hybridization and has a sequence such so as to permit interaction with certain agents, proteins, ribosomes, spliceosome, RBP (retinol-binding protein), miRNA (microRNA) and others present in the surrounding medium, e.g., within the cell where the bbASO is made to enter a cell.

By specific embodiments of this disclosure, the first nucleic acid strand is such that can specifically hybridize to a mutated target sequence, which may be a coding DNA sequence or an mRNA sequence and is typically complementary to that sequence: and the second strand is such so that after the first strand hybridizes to the target sequence, the exposed, second sequence, together with the non-hybridized portions of the full sequence, display a correct, non-mutated sequence. The first sequence will, typically, be a sequence complementary to the target sequence while the bases of the second sequence may be the same as those of the target sequence without a mutation. In the case of a single-point mutation, the first sequence will include, at the site of the point mutation, a base that is complementary to the aberrant base, while the second sequence will include a non-aberrant, i.e., the correct (non-mutated) base at the same position.

The two nucleic acid strands of the bbASO have typically an equal number of nucleobases. The number of nucleobases is at minimum a number that ensures that none of the strands of the complex is complementary to more than one RNA or DNA sequence (namely, to none other than the target sequence).

Provided by one aspect of the present disclosure is a nucleobases-comprising complex of one of the two following configurations. In the first configuration the complex comprising (i) a first strand comprising a first sequence of nucleobases and (ii) a second strand comprising a second sequence of nucleobases not complementary to said first sequence, the two stands being carried on a common backbone structure. As already noted above, the common backbone structure may be constituted by two coupled backbone structures, one being a first backbone structure of the first stand and the other a second backbone structure of the second strand. The first backbone structure and the second backbone structure may be bonded or coupled to one another to form the common backbone structure. The coupled backbone structure may also be a single backbone structure for the two strands. The nucleobases extend outwardly from the common backbone structure and, thus, the first sequence of nucleobases and the second sequence of nucleobases extend along two different, e.g., opposite, sides of the common backbone structure, thus, each of the base sequences is free to bond with a complementary nucleic acid sequence or free to interact with any other cellular material, molecules or molecular complexes, such as translation mechanism or transcription mechanism, or regulation mechanism, as the case may be.

It is to be noted that any combination of the described embodiments with respect to any aspect and any configuration of this present disclosure is applicable in different embodiments or aspects of this disclosures. In other words, any embodiment or aspects of the present disclosure can be defined by any combination of the described embodiments.

In some embodiments, as noted above, the bbASO is a single common backbone structure common for both strands. In another embodiments, as also noted above, the common backbone structure results from bonding two a priori separate backbones of each of the strands to form one common backbone structure.

In some embodiments, said first and said second backbone structures are sugar-based.

In some embodiments, said first strand and said second strand are nucleotide strands, including naturally occurring ribonucleotides or deoxyribonucleotides strand or non-naturally occurring nucleotides such as those mentioned above.

In some embodiments, said first strand and second strand are ribonucleotide strands. In some other embodiments said first strand and second strand are deoxyribonucleotide strands.

In some embodiments, said first strand can hybridize with a target sequence within a certain mRNA molecule, namely fully complementary to said target sequence. In some embodiments, the hybridization to the target sequence, e.g., in the mRNA or DNA, induces bonding conditions permitting the second strand to bind to or interact with certain agents, proteins, ribosomes, spliceosome, RBP (retinol-binding protein), miRNA (microRNA) and others present within the cell.

In some embodiments, said target sequence carries a mutation, namely said target sequence is transcribed from a mutated gene.

In some embodiments, said second strand is identical to that of a homologous target sequence that does not have said mutation, namely the translation thereof would result in a non-mutated, functional protein.

In some embodiments, said mutation is a point mutation and said second base sequence differs from the homologous sequence by one base at the site of said mutation.

In some embodiments, said second sequence is a transcription reading frame of full codons beginning in a complete codon.

In some embodiments, the first and second backbone structures are bonded to one another such as to obtain a desired alignment of bases between the first sequence and the second sequence, therefore obtaining an alignment between the first strand and the second strand. This further results in an alignment between the first strand, the second strand and the target sequence, when the first strand is hybridized thereto.

In some embodiments, said first sequence is a unique sequence intended to be complementary to the target sequence and not complementary to any other DNA or mRNA sequence.

In some embodiments, said first sequence comprises a first number of nucleobases and said second sequence comprises a second number of nucleobases different than the first number. Namely, the first and second sequences can be of different lengths.

In some embodiments, at least two adjacent nucleotides of the nucleobases of the first strand are bonded in a 5′-5′ or 3′-3′ linkage. In other words, at least one nucleotide in this strand is an inverted nucleotide that is bonded in a non-standard directionality to the next nucleotide in the sequence.

In some embodiments, at least two adjacent nucleotides of the nucleobases of the second strand are bonded in a 5′-5′ or 3′-3′ linkage. In other words, at least one nucleotide in this strand is an inverted nucleotide that is bonded in a non-standard directionality to the next nucleotide in the sequence.

In some embodiments, one of said two adjacent nucleobases in one or both of the first or the second stand is the last nucleotide of the sequence, either in the 3′-5′ or 5′-3′ direction.

In some embodiments, the common backbone structure is flexible to permit it to follow the twisted topology of an mRNA.

In some embodiments, the first backbone structure comprises phosphate-deoxyribose. Namely, the first strand is carried by a backbone similar to a standard DNA backbone.

In some embodiments, the second molecular strand is bonded to the phosphate of the phosphate-deoxyribose structure.

In some embodiments, the second backbone structure comprises phosphate-deoxyribose.

Yet another aspect of the present disclosure provides a method for compensating for a nucleic acid mutation in a target nucleic acid sequence. The method comprise forming a bbASO of this disclosure in which the first strand has a sequence of nucleobases that is complementary to the target sequence, whether the mutation is a single base or more than one base, and the second strand has a sequence of nucleobases comprises a correcting base sequence of said target sequence, or at least part of that sequence; and allowing the first strand to hybridize with said third base sequence.

The complex of this disclosure is useful, by some embodiments of the above method, for a personalized therapy for targeting mutated sequences and compensating for such genetic defects.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 is an illustration of a prior art ASO.

FIG. 2 is an illustration of a bbASO according to an embodiment of the present disclosure.

FIG. 3 is an illustration of an example of a sugar-based backbone structure carrying the nucleobases sequences of the complex.

FIGS. 4-5 are illustrations of uses of the nucleobases-comprising complex of the present disclosure to compensate a single-base mutation in an RNA sequence.

FIG. 6 is a schematic illustration of a non-limiting example of an embodiment of the complex of the present disclosure.

FIGS. 7A-7B are non-limiting examples of a complex according to the present disclosure, in which the two molecular strands are carried on a single common backbone structure.

### DETAILED DESCRIPTION

The following figures are provided to exemplify embodiments and realization of the invention of the present disclosure.

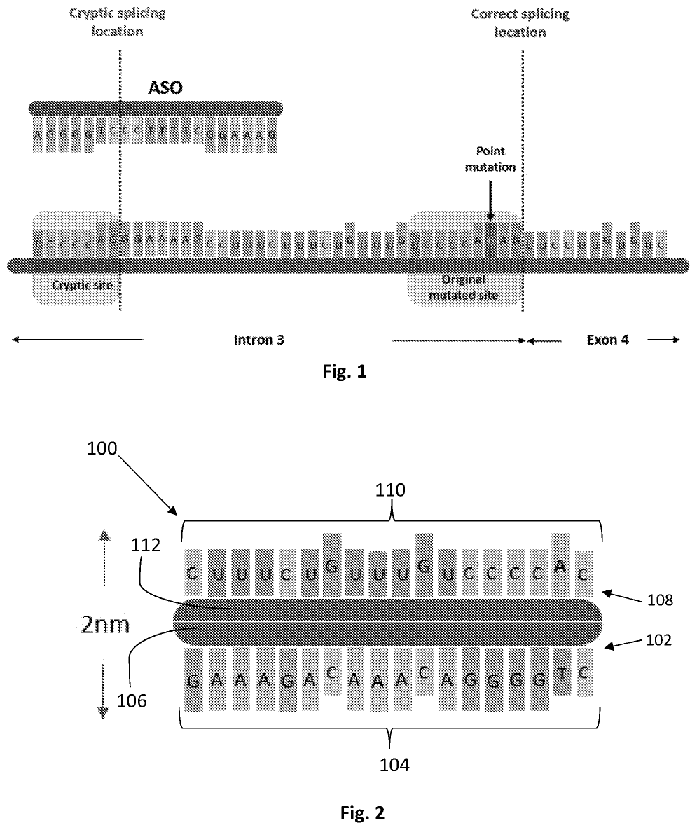

Reference is now being made to FIG. 1, which is an illustration of a use of an antisense oligonucleotide (ASO) as known in the art. The ASO, which is the upper sequence of the two, is designed to hybridize with a selected RNA sequence so as to block a cryptic site of a spliceosome such that it identifies the next most compatible site to splice, which is mutated in this example and is indicated as the original mutated site in the figure.

The present disclosure provides a new approach to deal with a mutation in an RNA or a DNA sequence. FIG. 2 is an illustration exemplifying a complex according to the present disclosure. The complex 100 comprises a first strand 102 that includes a first sequence of nucleobases 104 carried on a first backbone structure 106 and a second strand 108 that includes a second sequence of nucleobases 110 carried on a second backbone structure 112. The first and second backbone structures 106 and 112 are coupled to one another such that each of the first and second nucleobases sequences 104 and 110 is free to hybridize, bond or interact independently with a respective cellular material (DNA or RNA sequences, Ribosome, tRNA, etc.). The two molecular strands 102 and 108 contain an equal number of nucleobases and are aligned such that each nucleobase of the first strand is aligned with a respective base of the second strand. In that manner, each nucleobase of the second strand is practically replacing the nucleobase that is hybridized with the respective base of the first strand.

The first and the second nucleobases sequences can differ in one nucleobase. In this scenario, the first nucleobases sequence is complementary to an RNA sequence containing a single-base mutation and in the position of the nucleobase of the mutation, the second nucleobases sequence includes the correct nucleobase that is aligned with the mutated nucleobase.

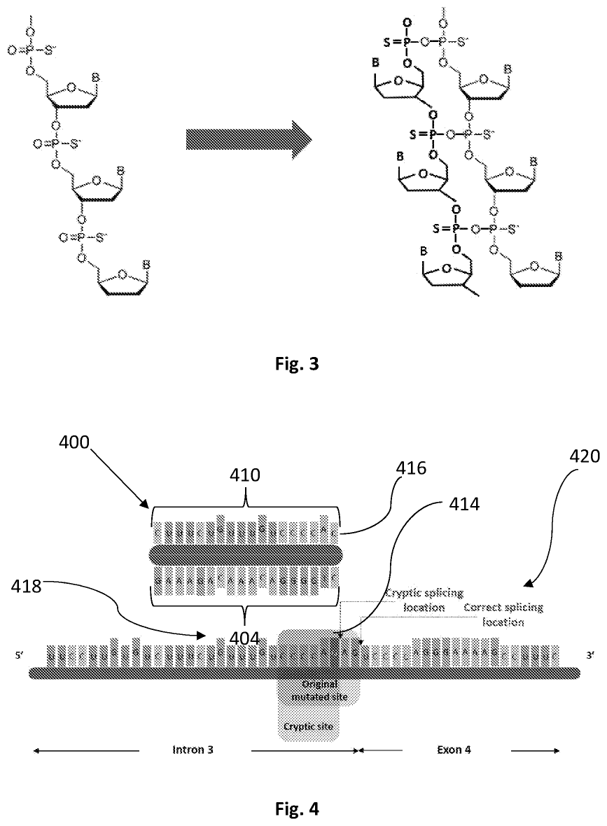

The backbones on which the nucleobases are being carried can be of different kind. For example, the backbones may be of the kind illustrated in FIG. 3, where the conjugation between the two strands is through the phosphate groups.

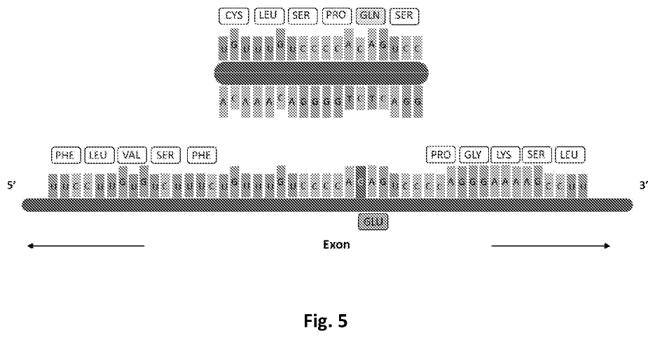

FIGS. 4 and 5 are illustrations that exemplify a compensation of a single-base mutation by the complex according to embodiments of the present disclosure. In the example of FIG. 4, the first nucleobases sequence 404 of the complex 400 hybridizes with a target sequence 418 of an RNA molecule 420 with a full transcribed sequence. Target sequence 418 comprises a mutated nucleobase 414. The second nucleobases sequence 410 includes a similar sequence to that of the target sequence 418, only differing in the position of the mutated nucleobase 414, which is the corrected nucleobase 416. Hybridization of the first sequence 404 to the target sequence 418 results in patching such that the combined sequence of the RNA molecule 420 and that of the second sequence 410 that is displayed is a fully corrected, i.e., not mutated sequence. In this example, the “replacement” of the mutated nucleobase restores the original splicing site of the RNA. In this case, the complex is designed so that the manipulated splicing site is constituted by a combination of nucleobases of the second nucleobases sequence and the original RNA sequence.

FIG. 5 exemplifies a scenario in which the first nucleobase sequence of the complex hybridizes with the mutation-comprising sequence portion in the RNA sequence such that the reading frame of the codons is not split between the original RNA sequence and the second nucleobases sequence. In other words, each codon is defined either by nucleobases of the original RNA sequence or by the second nucleobases sequence.

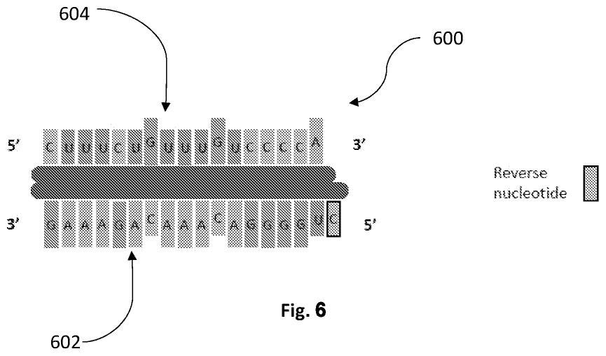

FIG. 6 is a schematic illustration of a non-limiting example of an embodiment of the complex of the present disclosure. The complex 600 includes a first strand 602 comprising a first sequence of nucleobases carried on a first backbone structure and a second strand 604 comprising a second sequence of nucleobases not complementary to said first sequence, carried on a second backbone structure. The first backbone structure and the second backbone structure are bonded to one another to form a coupled backbone structure. The two strands have different number of nucleobases. Furthermore, the first molecular strand comprises a reverse (or inverted) nucleotide, namely a nucleotide that is bonded to the next nucleotide in the sequence in a 5′-5′ linkage. In this example, the reverse nucleotide is the last nucleotide of the first molecular strand has a 3′-5′ directionality. The directionality of the molecular strand is defined by the majority of the bonds between the nucleotides, thus, although the directionality of the last nucleotide is changed to 5′-3′, the directionality of the entire molecular strand is considered to be 3′-5′. In this example, the last nucleotide on the 5′ end of the complementary strand mimics the function of the original C nucleotide which was mutated to G. It was added to the strand in “reverse” direction to allow its identification by the U2AF35 splicing factor as part of the YAG sequence of the pre-mRNA.

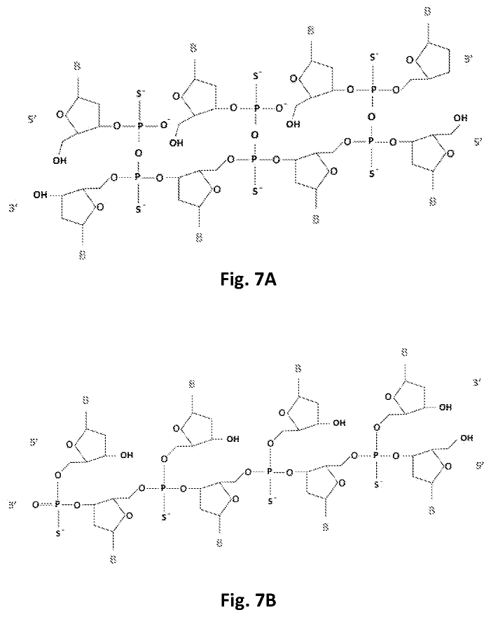

FIGS. 7A-7B are non-limiting examples of a complex according to the present disclosure, in which the two molecular strands are carried on a single common backbone structure. It is to be noted that each of the molecular strands can be hybridized to mRNA or any other RNA or DNA molecule or bonded to other cellular complexes such as RNA binding proteins.

Exemplary synthesis schemes for the production of a complex of this disclosure are described below.

Exemplary synthesis schemes for the production of a complex of this disclosure are described below.

The exemplary synthetic procedure includes the preparation of three oligonucleotide building blocks consisting of two nucleosides linked via phosphate moiety (as shown below).

These building blocks may be further conjugated one to another creating a desired “two stranded” oligonucleotide sequence (as shown below). Some synthesis parameters relevant for this exemplary procedure may be found, for example, in Dikshit et al (Canadian Journal of Chemistry. 66 (12): 2989-2994), or Ferris JP et al (Origin of Life and Evolution of Biospheres) 2011 June; 41 (3): 213-36.

DNA bases may be used for the synthesis instead of RNA bases owing to higher relative stability.

The first building block is synthesized by reacting of deoxycytidine phosphate and acetic anhydride in pyridine to result in compound 1. The acetate group protects the exocyclic amine and 3′-hydroxyl groups of deoxycytidine phosphate.

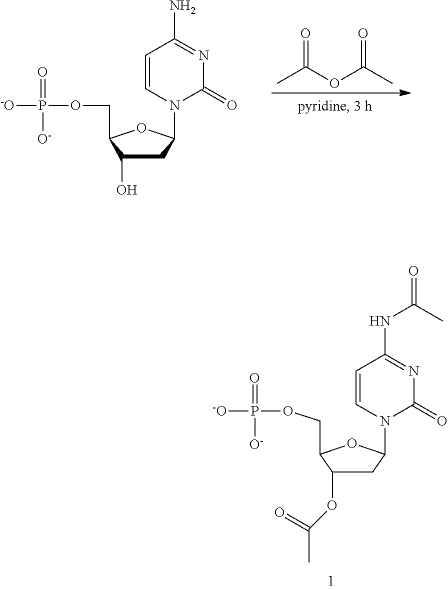

Deoxyadenosine is then protected twice by orthogonal protecting groups to obtain compound 3. The exocyclic amine reacts solely with naphthalic anhydride (can be removed by basic conditions) and 5′-hydroxyl group is protected by dimethoxytrityl chloride (can be removed by mild acidic conditions).

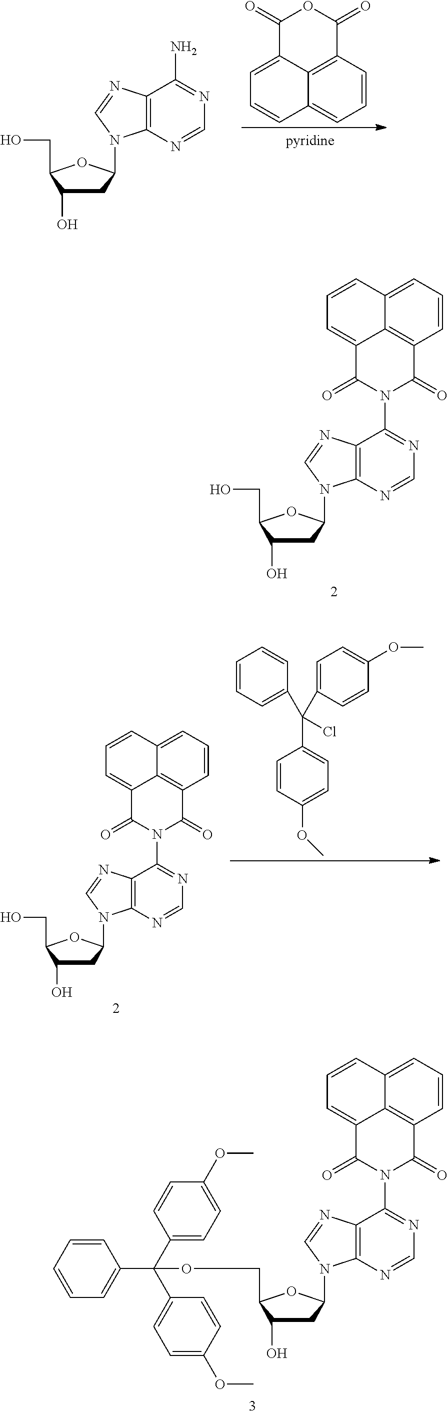

The conjugation of two compounds 1 and 3 is performed using 2,4,6-triisoprpylbenzenesulfonyl chloride. 2,4,6-Triisoprpylbenzenesulfonyl chloride activates the phosphate moiety of compound 1 and allows nucleophilic attack of 3′-hydroxyl group of compound 3. The obtained compound 4 is then subjected to purification, for example through cellulose ion exchange column. The isolated compound 4 is then treated with mild acidic conditions to remove dimethoxytrytil protecting group from 5′-hydroxyl group of the obtained dimer 5.

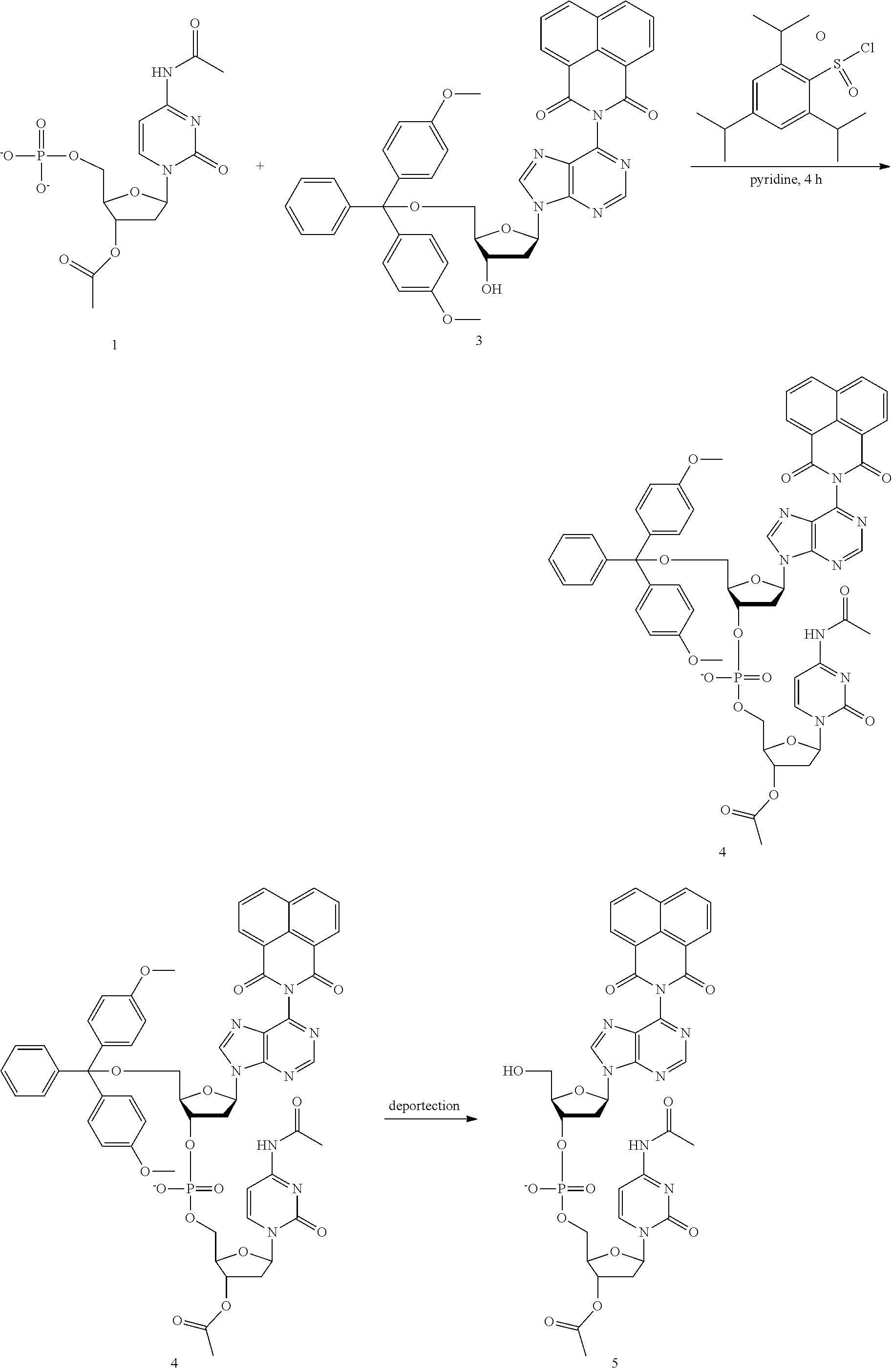

Second building block preparation begins with the protection of 5′-hydroxyl of uridine with dimethoxytrityl chloride to obtain compound 6. Compound is reacted with compound 1 in the presence of 2,4,6-triisoprpylbenzenesulfonyl chloride to obtain the second building block compound 7. Compound 7 can be isolated using cellulose ion exchange column.

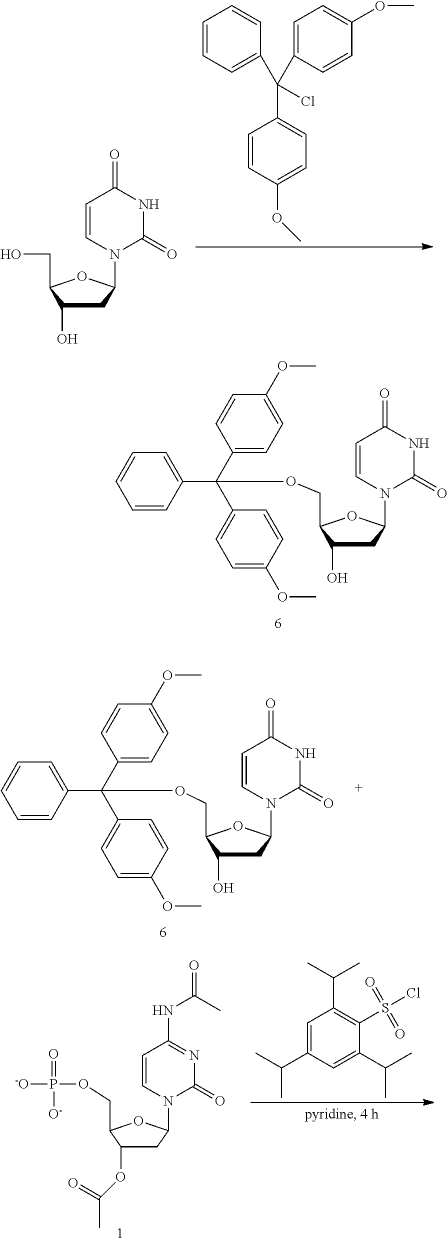

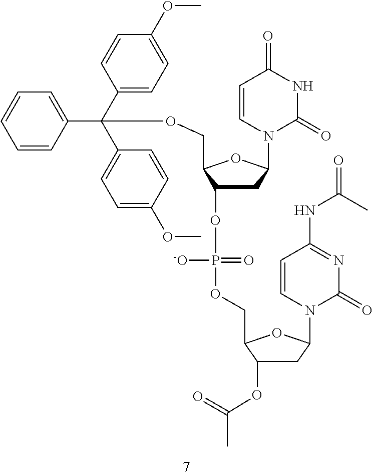

Further coupling of the building blocks is presented below.

The unprotected 5′-hydroxyl group of compound 5 attacks the phosphate group of compound 7 in the presence of coupling reagent 2,4,6-triisoprpylbenzenesulfonyl chloride. The tetramer product 8 is purified, e.g. by ion exchange column, followed by removal of the dimethoxytrityl group by mild acidic condition to expose free 5′-hydroxyl group, resulting in conjugate 9 for further nucleophilic attack of the next dimer building block.

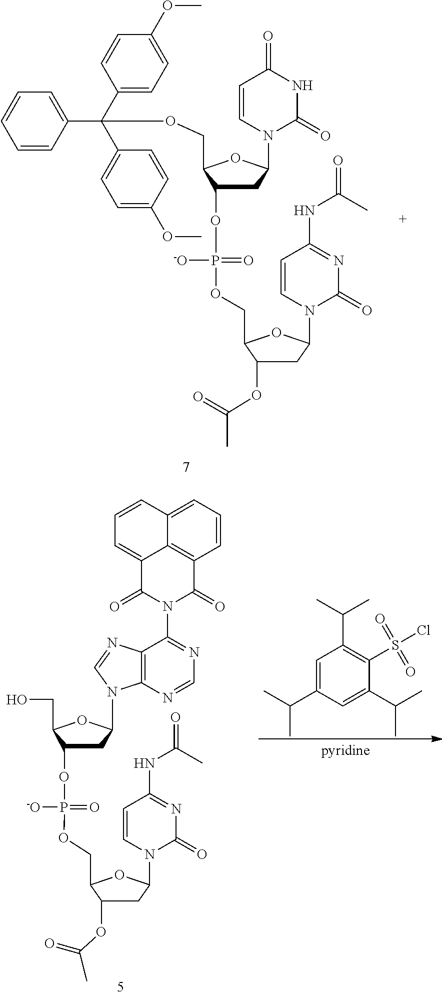

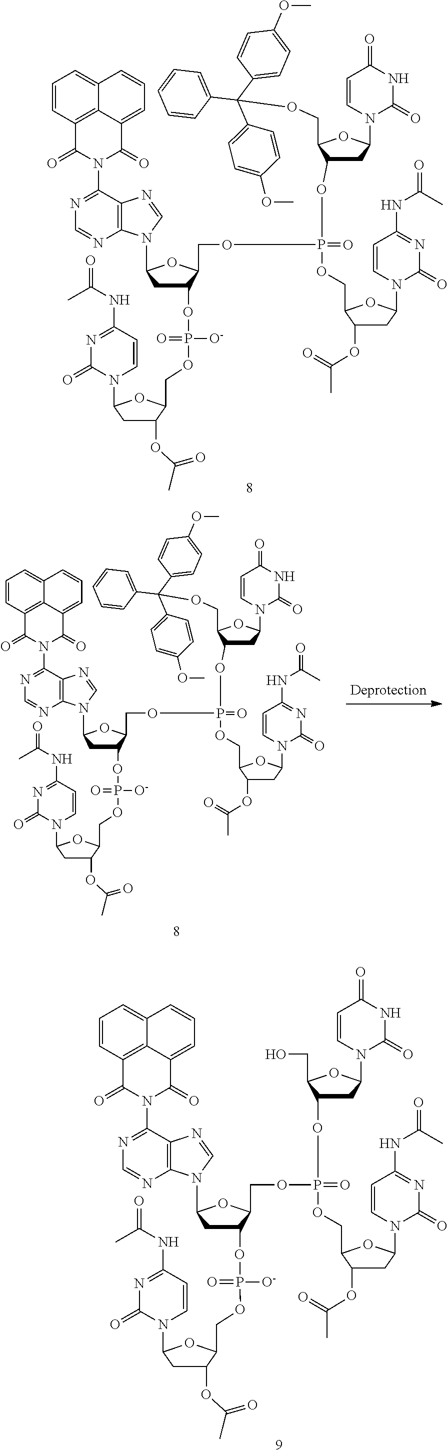

The pure tetramer 9 can be reacted with an additional building block. i.e. compound 7. using the same coupling reaction to result in compound 10.

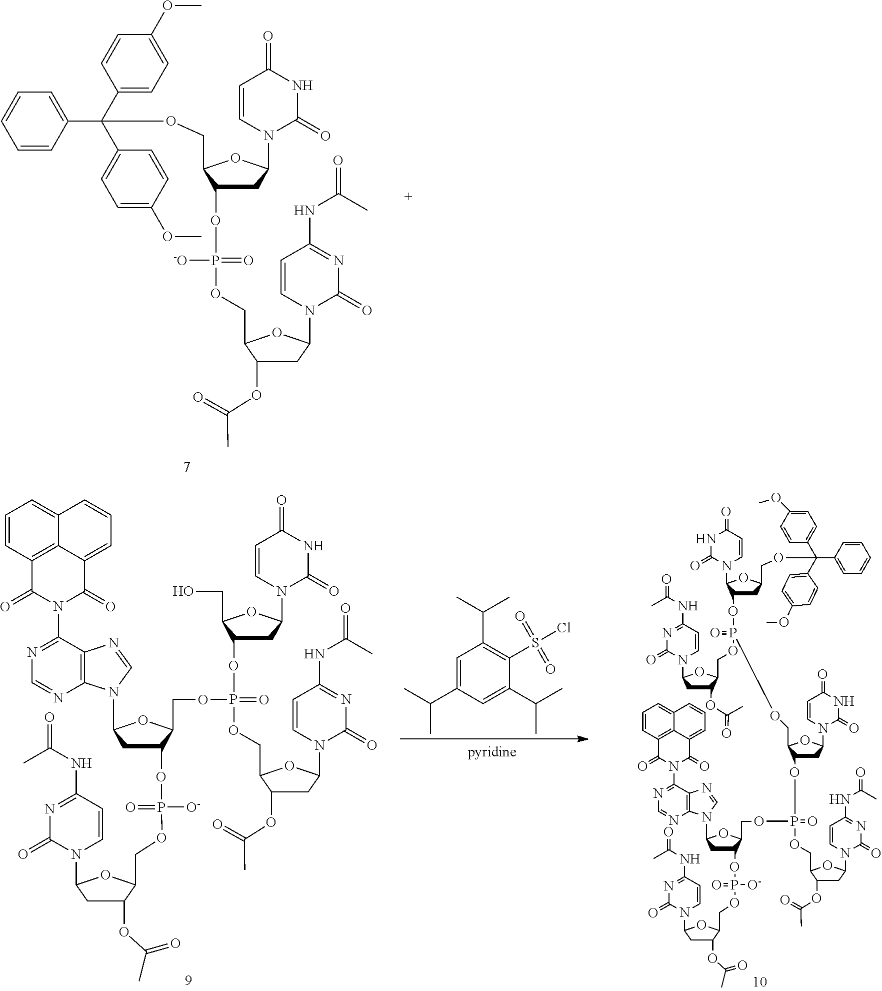

Obtained hexamer product can be purified by ion exchange chromatography and the isolated product is then treated by ammonia in methanol to remove acetate and naphthalic protecting groups to obtain conjugate 11.

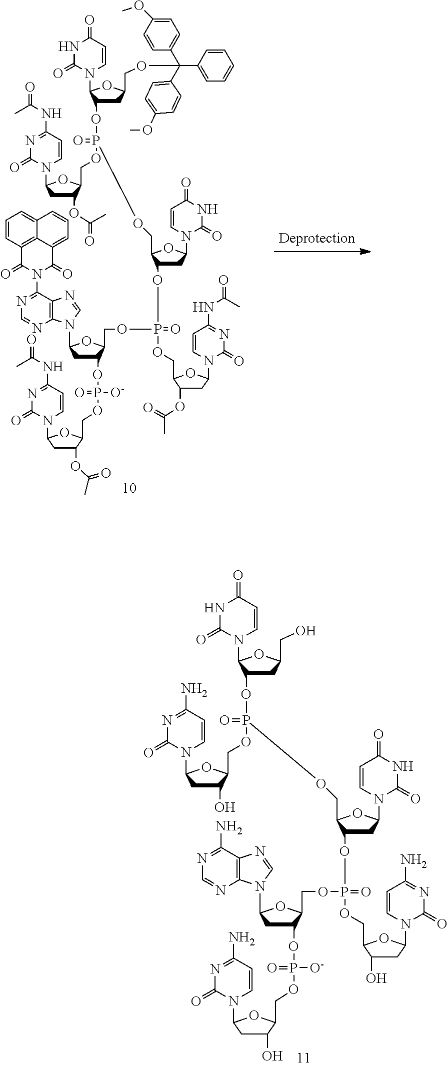

The linear structure of hexamer conjugate 11 is shown below.

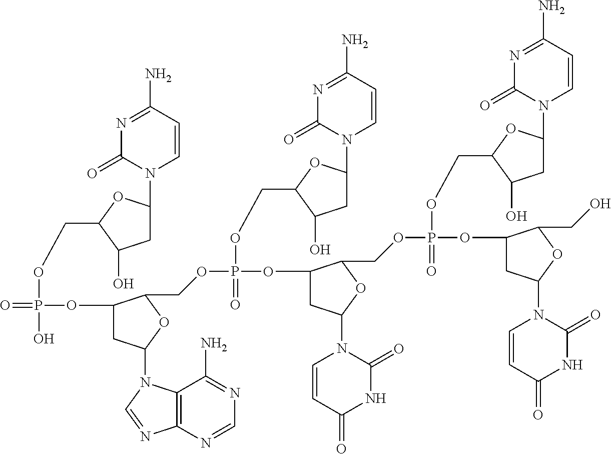 [SMILES: Nc1ccn(C2CC(O)C(COP(=O)(O)OC3CC(n4cnc5ncnc(N)c54)OC3COP(=O)(OCC3OC(n4ccc(N)nc4=O)CC3O)OC3CC(n4ccc(=O)[nH]c4=O)OC3COP(=O)(OCC3OC(n4ccc(N)nc4=O)CC3O)OC3CC(n4ccc(=O)[nH]c4=O)OC3CO)O2)c(=O)n1]

1. A nucleobases-comprising complex, comprising:
a first strand comprising a first sequence of nucleobases;
a second strand comprising a second sequence of nucleobases not complementary to said first sequence;
the two strands being carried on a common backbone structure;
said first sequence of nucleobases and said second sequence of nucleobases extend along two sides of the common backbone structure.

2. The complex of claim 1, wherein the coupled backbone structure is constituted by two coupled backbone structures, one being a first backbone structure of the first stand and the other a second backbone structure of the second strand, bonded to one another to form a coupled backbone structure.

3. The complex of claim 1, wherein the coupled backbone structure comprises a single backbone structure common to the first and the second strand.

4. The complex of claim 1, wherein said first and said second backbone structures are sugar-based.

5. The complex of claim 1, wherein said first strand and said second strand are nucleotide strands.

6. The complex of claim 1, wherein said first strand and said second strand is constituted by ribonucleotides.

7. The complex of claim 1, wherein said first strand can hybridize with a target sequence within a specified mRNA molecule.

8. The complex of claim 7, wherein said target sequence carries a mutation;
wherein said second strand is identical to that of a sequence similar to the target sequence but not having said mutation;
wherein said mutation is a point mutation and said second base sequence differs from the target sequence by one base at the site of said mutation.

9-10. (canceled)

11. The complex of claim 8, wherein said second strand is a transcription reading frame of full codons beginning a complete codon.

12. The complex of claim 7, having a desired alignment of bases between the first and second strands.

13. The complex of claim 1, wherein said first strand is a unique sequence complementary to only one nucleobases sequence.

14. The complex of claim 1, wherein said first strand comprises a first number of nucleobases and said second strand comprises a second number of nucleobases different than the first.

15. The complex of claim 1, wherein at least two adjacent nucleotides of the first strand are bonded in a 5′-5′ or 3′-3′ linkage.

16. The complex of claim 1, wherein at least two adjacent nucleotides of the second strand are bonded in a 5′-5′ or 3′-3′ linkage.

17. The complex of claim 16, wherein one of said two adjacent nucleotides is the last nucleotide of the sequence.

18. The complex of claim 1, wherein said first backbone structure comprises phosphate-deoxyribose.

19. The complex of claim 18, wherein the second strand is bonded to the phosphate of the phosphate-deoxyribose structure.

20. The complex of claim 1, wherein said second backbone structure comprises phosphate-deoxyribose.

21. A method for compensating for a nucleic acid mutation in a target nucleic acid sequence, the method comprising:
forming a nucleobase-comprising complex comprising a first strand with a first sequence of nucleobases that is complementary to a target base sequence, a second strand with a second sequence of nucleobases not complementary to said first sequence and comprises a correcting base sequence to that of the target sequence, the two strands being carried on a common backbone structure, said first sequence of nucleobases and said second sequence of nucleobases extending along sides of the common backbone structure; and
allowing the first strand of the complex to hybridize with said target sequence.

22. A method for compensating for a nucleic acid mutation in a nucleic acid sequence, the method comprising:
allowing a complex of claim 1, wherein the sequence of the first strand is complementary to a target base sequence that comprises the mutation and the second strand has a sequence of nucleobases that comprises a correcting base sequence to that of said target sequence, to hybridize with said target sequence.
